(19) United States
(12) Patent Application Publication
(10) Pub. No.: US 2023/0094121 A1
(43) Pub. Date: Mar. 30, 2023

(54) SOIL MOISTURE DETECTION SENSOR HAVING METAL-ORGANIC FRAMEWORK AND METHOD

(71) Applicant: KING ABDULLAH UNIVERSITY OF SCIENCE AND TECHNOLOGY, Thuwal (SA)

(72) Inventors: Mohamed EDDAOUDI, Thuwal (SA); Khaled N. SALAMA, Thuwal (SA); Osama SHEKHAH, Thuwal (SA); Sandeep G. SURYA, Thuwal (SA); Saravanan YUVARAJA, Thuwal (SA)

(21) Appl. No.: 17/910,138

(22) PCT Filed: Mar. 9, 2021
(86) PCT No.: PCT/IB2021/051949

§ 371 Date: Sep. 8, 2022

Related U.S. Application Data
(60) Provisional application No. 62/987,521, filed on Mar. 10, 2020.

(51) Int. Cl. G01N 27/22; G01N 33/24

(52) U.S. Cl. CPC G01N 27/223; G01N 27/226; G01N 33/246; G01N 2033/245

(57) ABSTRACT

A moisture sensor is configured to be deployed in soil for measuring a moisture content. The moisture sensor includes a housing; a transistor configured to interact with water from the soil; a power source configured to generate an electrical current; and a processing unit configured to receive a reading from the transistor, and to calculate the moisture content of the soil based on the reading. The transistor includes a metal-organic framework, MOF.

### CROSS-REFERENCE TO RELATED APPLICATIONS

This application claims priority to U.S. Provisional Patent Application No. 62/987,521, filed on Mar. 10, 2020, entitled “DETECTION OF SOIL MOISTURE BY IMPEDANCE AND TRANSISTOR SENSORS COATED WITH METAL-ORGANIC FRAMEWORKS,” the disclosure of which is incorporated herein by reference in its entirety.

### BACKGROUND

### Technical Field

Embodiments of the subject matter disclosed herein generally relate to a moisture detecting sensor, and more particularly, to building a moisture detecting sensor that uses a metal-organic framework for enhancing the moisture detection.

### Discussion of the Background

As the fresh water reserves are being depleted faster than ever, for any country that uses a large amount of fresh water for farming there is a need for precise irrigation management, where optimum irrigation plays a vital role. There is a significant absence of advanced technology in the dry farming regions, which leads to a decrease in the crop yield. For precise irrigation management (optimum irrigation), it is necessary to monitor and maintain the moisture levels to increase the crop yield. For optimum irrigation, soil moisture sensors are widely used. There is ongoing research to understand the evapotranspiration of the plants in an agro-ecosystem, which also helps the scientists in understanding the plant's biology, disease identification, water uptake, and light wavelength tolerability. Still, there is a shortage of sensor technology in the field of agriculture, where significant contributions need to be addressed with a multi-disciplinary approach.

Soil matrix is the mixture of different organic contaminants, minerals, nutrients, metals, etc. The irrigation of a given crop is dependent not only on that crop, but also on the soil matrix. Thus, an analysis of the optimum irrigation based on the specific soil matrix is necessary. In the field of agriculture, to attain optimum irrigation, the moisture in the soil needs to be maintained between the field capacity (FC) and the wilting point (WP) with respect to time. This helps in the conservation of water as well as in the increase in crop yield. FC is a state of the soil, in which crops have a sufficient intake of water. The WP is the point that indicates that crops need water. The permanent wilting point (PWP) is the point that indicates water deficiency in the soil, and saturation is the stage at which water is present in excess in the soil.

One of the basic needs of the plant/tree is the water content in the soil that they can absorb to survive in the vadose zone. To understand the growth of the plants and to avoid potential dangers in different crops, it is desired to keep track of the soil moisture content. Apart from the above, other factors that influence the growth of the plants are soil pH, soil nutrients, and temperature. However, the soil moisture appear to be the highest factor in the hierarchy of factors that influence a crop. Thus, determining the soil moisture in an accurate way and with an inexpensive device is desired.

The soil moisture can be determined by two different techniques, namely volumetric and gravimetric measurements. For the former technique, there exist technologies to perform these tasks like time domain reflectometry (TDR), neutron scattering probe, frequency domain reflectometry (FDR), heat-pulse, and the resistive method. The first three techniques are expensive and complex whereas the last two techniques are simple to use and affordable, but they need soil specific calibration and have a high-response time.

Micro-electro-mechanical systems (MEMS) platform is another alternative that is built-on Si substrates, which consume very low power and has the ability to deliver affordable sensors. Moreover, micro-cantilevers from the MEMS family are apt for sensing gas, moisture, humidity, and bio-analytes with different transduction capabilities, [1] which used a piezo-resistive cantilever to detect moisture in soil with the help of a polymer called polyaniline. Similarly, [2] used the micro-cantilever platform for soil moisture sensing applications. In the case of the piezo-resistive cantilever reported in [1], the fabrication process is complex (multi-level lithography), and the sensitivity of the sensor depends on the thickness of the sensing element and its burial depth.

Resistive and transistor devices, on one hand, are simple to build with the help of interdigitated electrode structures (IDEs) on multiple substrates [3, 4]. Nevertheless, they are prone to temperature drifts, other disadvantages include a high-response time (in hours), and limited shelf life. On the other hand, capacitance-based sensing devices are more immune to temperature drifts and their response time is typically in minutes. The fabrication process of IDEs is also not as complex as MEMS and in addition, an IDE can be swept with a band-width of frequencies to tune the sensitivity. The advantage of IDE structures is that they can be realized with both cleanroom and non-clean room processes like laser engraving, inkjet printing, etc. Additionally, because the water molecules have a high relative permittivity (˜80) compared to air, the sensitivity of the sensor is improved.

However, the existing sensors suffer from one or more problems associated with their sensitivity, selectivity and affordability, when deployed for in-situ agriculture applications. Thus, there is a need for a new sensor that is capable of overcoming one or more of the above discussed deficiencies while being inexpensive and accurate.

### BRIEF SUMMARY OF THE INVENTION

According to an embodiment, there is a moisture sensor configured to be deployed in soil for measuring a moisture content. The moisture sensor includes a housing, a transistor configured to interact with water from the soil, a power source configured to generate an electrical current, and a processing unit configured to receive a reading from the transistor, and to calculate the moisture content of the soil based on the reading. The transistor includes a metal-organic framework, MOF.

According to another embodiment, there is a moisture sensor configured to be deployed in soil for measuring a moisture content. The moisture sensor includes a housing, a capacitor configured to interact with water from the soil, a power source configured to generate an electrical current, and a processing unit configured to receive a reading from the capacitor, and to calculate the moisture content of the soil based on the reading. The capacitor includes a metal-organic framework, MOF, as a dielectric material.

According to yet another embodiment, there is a method of making a moisture sensor for measuring a moisture content in soil. The method includes providing an electronic element that includes a metal-organic framework, MOF, providing a power source configured to generate an electrical current, connecting a processing unit to the electronic element and the power source, and configuring the electronic element to receive a reading from the electronic element and to calculate the moisture content of the soil based on the reading.

### BRIEF DESCRIPTION OF THE DRAWINGS

Fora more complete understanding of the present invention, reference is now made to the following descriptions taken in conjunction with the accompanying drawings, in which:

FIG. 1 is a schematic of the chemical structure of a Zr-fum-fcu-MOF;

FIG. 2 illustrates a transistor that uses the Zr-fum-fcu-MOF;

FIG. 3 illustrates a capacitor that uses the Zr-fum-fcu-MOF;

FIG. 4 illustrates a moisture sensor that uses the transistor of FIG. 2 or the capacitor of FIG. 3;

FIG. 5 illustrates the capacitance response of the Zr-fum-fcu-MOF;

FIG. 6 illustrates the sensitivity of the moisture sensor that uses the Zr-fum-fcu-MOF;

FIG. 7 illustrates the response time of the moisture sensor that uses the Zr-fum-fcu-MOF;

FIG. 8 illustrates the temperature stability of the moisture sensor that uses the Zr-fum-fcu-MOF; and

FIG. 9 is a flow chart of a method for making the moisture senor that uses the Zr-fum-fcu-MOF.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a transistor or capacitor-based sensor that uses a Zr-based metal-organic framework (MOF) for enhancing moisture absorption, sensitivity and selectivity. However, the embodiments to be discussed next are not limited to the Zr-based MOF, but may be applied to other MOFs.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

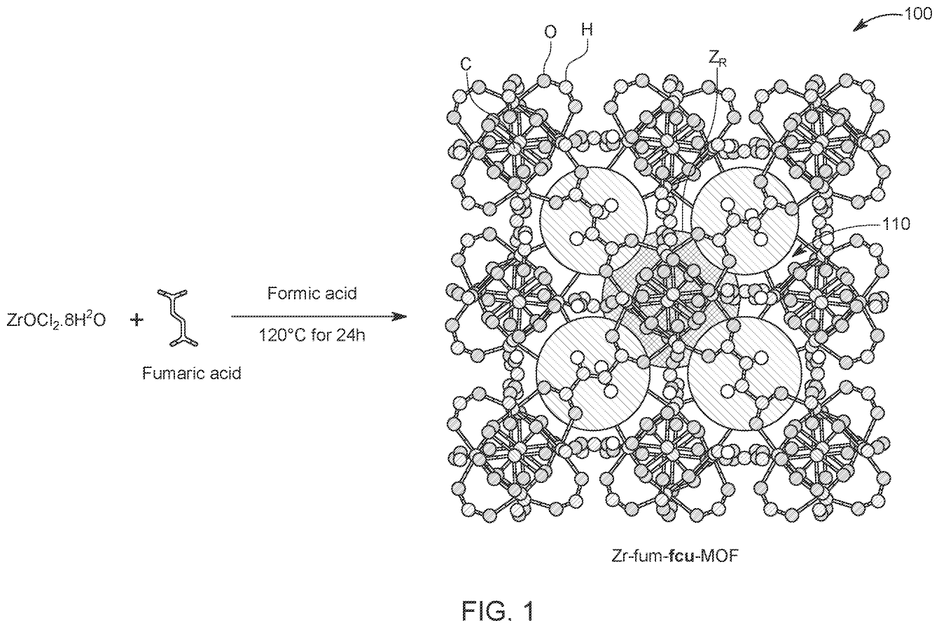

According to an embodiment, a moisture detection sensor uses a Zr-based MOF (which is called herein MOF-C or Zr-fum-fcu-MOF) having a high-surface area. The MOF-C has the chemical structure 100 shown in FIG. 1, which translates into the chemical composition of Zr₆O₄OH₄(C₄H₂O)₆. The MOF-C 100 is generated by mixing zirconium chloride octahydrate ZrOCl₂.8H₂O with fumaric acid HO₂CCH═CHCO₂H in the presence of formic acid HCOOH for about 24 h at a temperature of about 120° C. The connection of metal ions or clusters with multi-topic organic linkers creates a regulated nano-space 110 within the extended crystalline structure of the MOF-C 100. The guest molecules can be incorporated into the nano-space 110 of the MOF 100 through their molecular sieving effects, π-π interaction, hydrogen bonding, and electrostatic interactions, etc. The nano-space/functionality in the MOF-C can recognize the guest molecules via different mechanisms like interactions, size and shape. These tunable features of the MOF-C in addition to its high surface-areas make it a good fit for the moisture sensor. The MOF-C was designed to be hydrolytically stable and highly porous, affording exceptional adsorbed water uptake.

The MOF-C material when used with a capacitor or transistor has been found to be more sensitive and selective to moisture then materials such as polymers, transition metal chalcogenides, quantum dots, and carbonaceous compounds. Although the polymers have a tunable pore size, they suffer from degradation problems similar to other 2D materials and quantum dots, which are also expensive and difficult to process.

In one embodiment, the deposition of the hydrolytically stable MOF-C is achieved directly on an interdigitated electrodes (IDEs) substrate, which allows the resulting sensor to sense a change in the sensing film permittivity upon diffusion/adsorption of the targeted analyte. In this case, impedance sensors were selected because of their simple structure, compatibility with standard CMOS technology and their ability to operate normally at room temperature, for assisting low-power applications. Besides, impedance (capacitive and/or resistive) sensors enable miniaturization, as they are reliably and inexpensive.

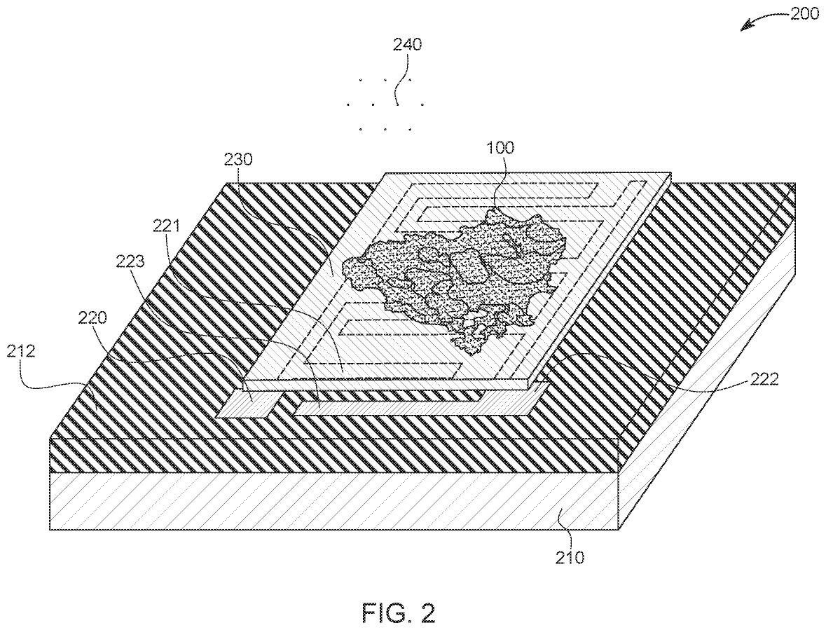

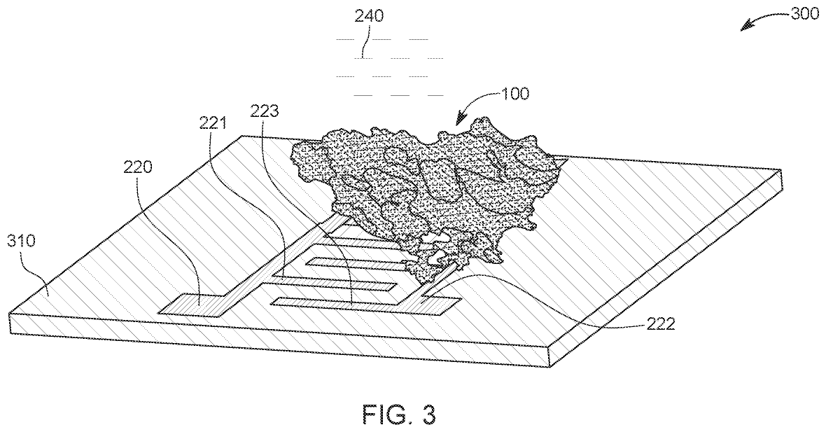

The MOF-C material may be used to improve the moisture detection of a transistor as shown in FIG. 2 or a capacitor as shown in FIG. 3. Each of these devices are then used for moisture sensing, thus, being the base of the moisture sensor. The transistor 200 has a gate 210 (for example, made of Au) on which a dielectric material 212 is located. IDE electrodes 220 and 222 are located over the dielectric material 212 and each IDE electrode has plural fingers 221 and 223 that are interleaved as shown in the figure. The IDE electrodes 220 and 222 may be made of Ti and Au, or any other conductor material. The IDE electrodes act as a source and drain in this embodiment. A semiconductor layer 230 is located over the dielectric material 212 and over the IDE electrodes 220 and 222 so that at least one finger 221 of the IDE electrode 220 and at least one finger 223 of the IDE electrode 222 are fully covered (i.e., fully sandwiched between the semiconductor layer 230 and the dielectric material 212). The MOF-C 100 is placed over the semiconductor layer 230 so that the MOF-C 100 directly interacts with the moisture (water droplets) 240 in the ambient. Due to its structure discussed above, the MOF-C 100 directly adsorbs the moisture 240 from the ambient, thus changing the electrical properties of the semiconductor layer 230. This change can be detected, as discussed later, and mapped to the amount of moisture in the ambient.

FIG. 3 shows a capacitor 300 that uses the MOF-C 100 as the dielectric material. The capacitor 300 has a substrate 310, which may be Si, SiO, or any other material that is not a conductor. Similar IDE electrodes 220 and 222 as for the transistor 200 are located over the substrate 310, with at least one finger of each being covered by the MOF-C 100. The electrical field that extends from one finger to another finger enters through the MOF-C 100. Because the MOF-C's electric permeability changes with the amount of moisture 240 absorbed from the ambient, the capacitance of the capacitor 300 changes with the moisture amount. Because the MOF-C 100 is very selective and very sensitive to moisture, the capacitor inherits the same properties.

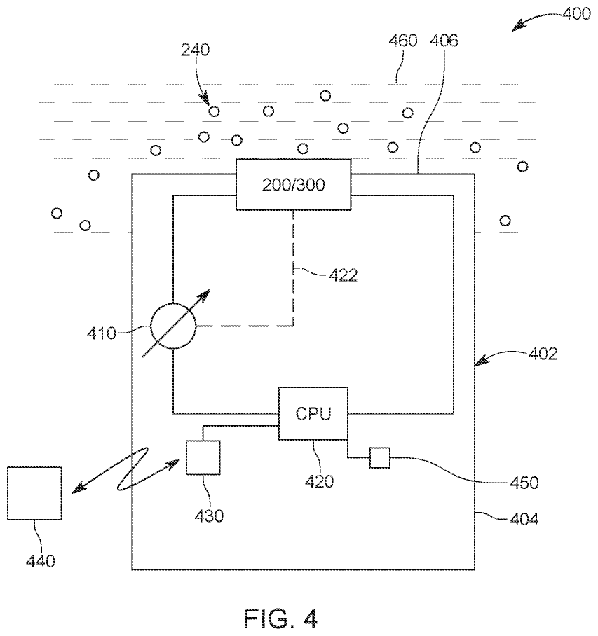

FIG. 4 shows a moisture sensor 400 that uses either the MOF-C based transistor 200 or the MOF-C based capacitor 300 previously discussed. The sensos also includes a power source 410, for example, a battery, and a processing unit 420 (for example, an integrated circuit) for controlling the power source 410, and also for processing the current or voltage experienced by the transistor 200 or capacitor 300 when the moisture enters the MOF-C 100. If the transistor 200 is used, an additional current path 422 is used to connect it to the gate of the transistor. Although FIG. 4 shows the current path 422 connecting the gate of the transistor 200 to the power source 410, other connections may be made and further electrical elements may be added.

The processing unit 420 may include additional electronics for transforming the DC current of the power source 410 into AC current having a given frequency, e.g., 500 Hz, and applying this current to the capacitor 300 or transistor 200 for measuring their capacitance. The processing unit 420 may also be linked to a transmitter or transceiver 430, which is configured to send the moisture readings from the sensor to an external device 440, for example, a smartphone. Other external devices may be used, for example, a cell tower, a WI-Fl device, etc. The transmitter or transceiver may be selected to use any known radio-frequency (RF) or any known communication channel.

The components of the sensor 400 may be placed into a housing 402 to protect them from the soil particles. In this regard, note that the moisture sensor 400 is configured to be used in the ground, i.e., to be at least partially buried in the soil so that the sensitive element (transistor 200 or capacitor 300) can receive the water particles from the soil. However, a part of the transistor 200 or capacitor 300 is allowed to directly interact with the soil 460, i.e., the moisture 240 can directly interact with the MOF-C 100 to change its electrical permeability, thus, affecting the electrical characteristics of the sensor. This can be achieved by having the housing 402 made of a first part 404 that is impermeable to water or other soil parts, for protecting the electronics from any direct interaction with the soil 460, and a second part 406, which is selected to allow moisture to pass it, for example, a nylon mesh having holes with a size around 150 μm. The second part 406 allows the moisture 240 to enter the housing and reach the MOF-C 100, but does not allow the soil particles 460 to enter the housing. Other materials may be used for the housings as known in the art of moisture sensors.

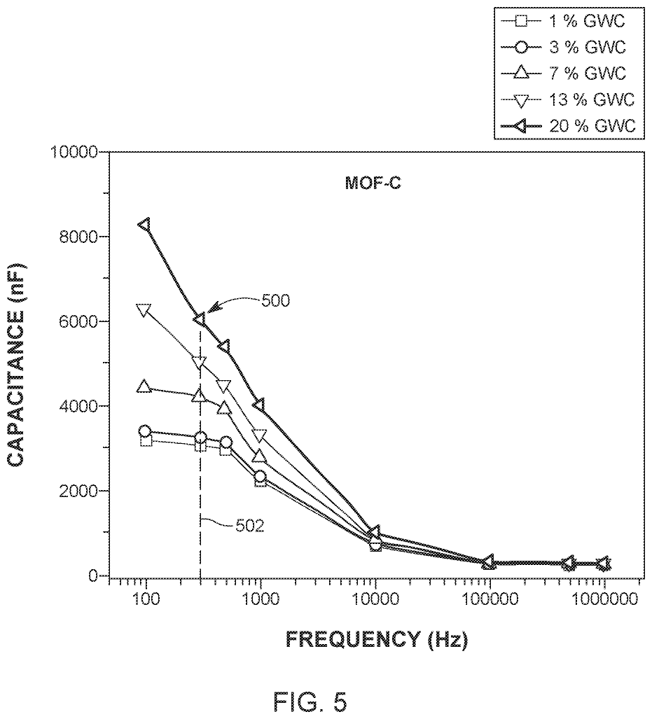

The characteristics of the MOF-C 100 have been studied as now discussed. FIG. 5 shows the capacitance response of the MOF-C 100 for a given soil for various gravimetric water content (GWC) percentages. The GWC is indicative of the amount of water retained by a given soil. The capacitance C, which is plotted on the Y axis in FIG. 5, was measured with an LCR meter in the frequency range of 100 Hz to 2 MHz with a voltage bias of 1 V. The measurements were conducted at constant temperature and humidity of about 25° C. and 50% RH, respectively. The water content of the tested soil has varied between 1 and 20% GWC. The changes in the sensor capacitance when exposed to the various GWC percentages indicate that the capacitance decreases with the increase in frequency. The reason for this could be that the direction of the electrical field varies rapidly with an increase in the frequency, and the polarization of the adsorbed water does not catch up with this high-frequency rate and hence the dielectric constant is small and becomes independent of the soil moisture. Thus, among all tested frequencies, the 500 Hz was selected for the other experiments, as it proved to be quantitatively sensitive.

FIG. 5 also indicates that the sensor capacitance monotonically increases with an increase in the soil water content. An increase in the soil water content increases the adsorbed water molecule at the MOF-C surface, which strengthens the polarization and thus the capacitance of the sensor increases. FIG. 5 also shows that the sensor's capacitance showed a minimal variation (error bar 500) and the MOF-C is highly sensitive as it shows a 200% increase (along line 502) in sensitivity for the 500 Hz when comparing the 1% to 20% GWC configurations.

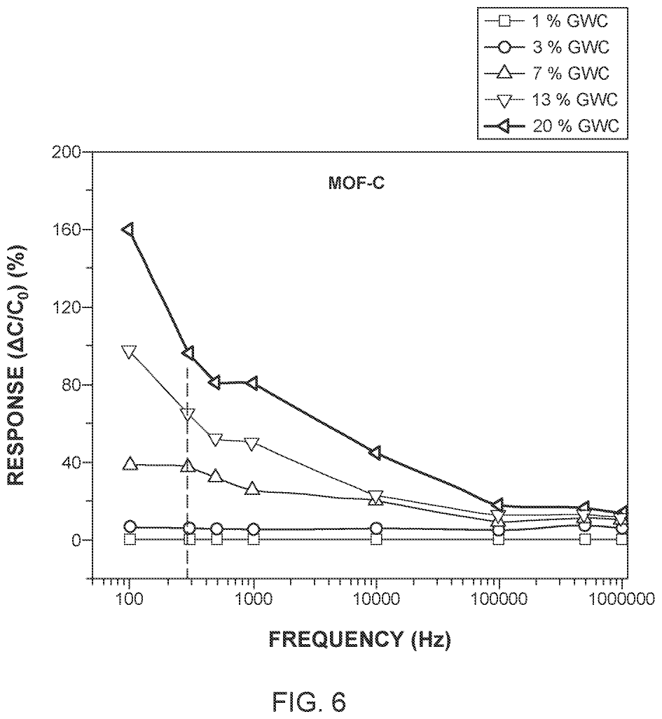

FIG. 6 also shows that the sensitivity of the sensor 400 is high when the relative capacitance is measured. The relative capacitance is defined as the ratio between (1) the change in capacitance ΔC from a reference value C₀ (for example, dry soil with no moisture) to a current value C_{c}, and (2) the reference value C₀. The change in the relative capacitance of the sensor 400 when measured for various frequencies for various GWC soil samples shows that for most of the frequencies, the readings for various GWC percentages are well separated, which means that the sensor can distinguish between soils having moisture values close to each other. This also means that the sensitivity of the sensor is high. Note that at 500 Hz, the relative capacitance for 1% GWC is about zero and for the 20% GWC is about 90, which means that the readings of the GWC values are well separated from each other.

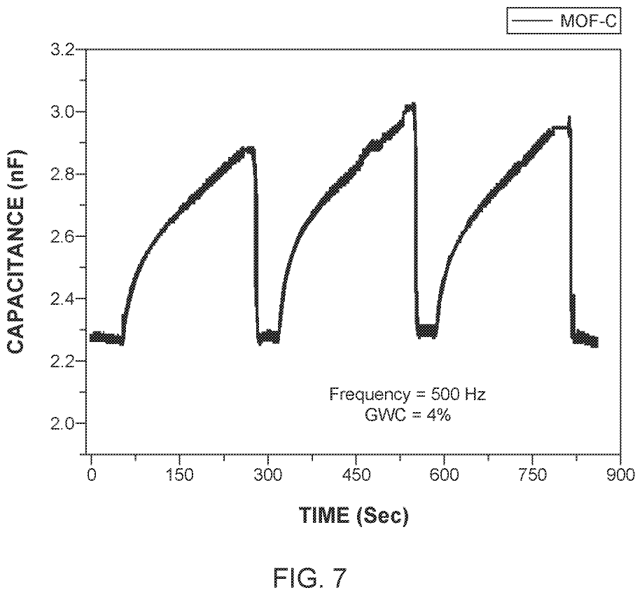

Real-time screening of moisture content in soil is of utmost importance to ward off potential dangers like the growth of fungi and other pests for the various crops. The MOF-C based sensor 400 proved to be suitable for the detection of the soil moisture in the soil with a reasonable response and recovery time at 4% gravimetric moisture content. In the case of the soil used above for the various measurements, as shown in FIG. 7, to reach 90% of the maximum response, the MOF-C took about 200 s, whereas the recovery time is about 25 s, which are appropriate for a real time measurement in the field.

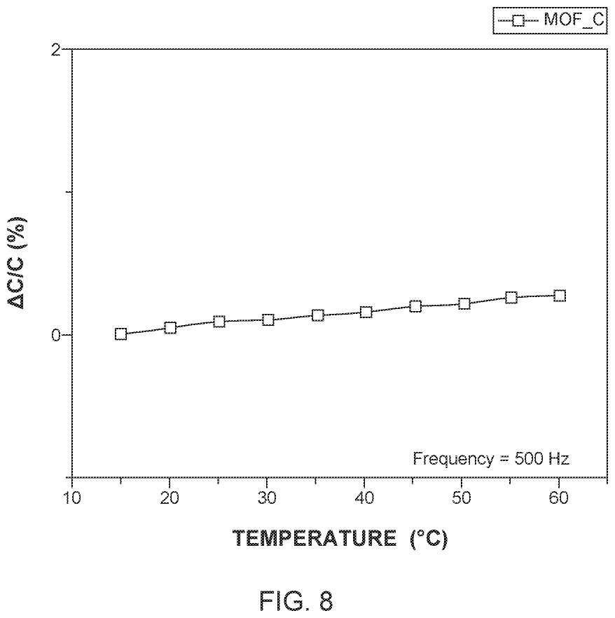

When a moisture sensor is used in the field, especially in areas where the sun is very intense, there is the concern that the moisture sensor can generate inaccurate readings due to the high ambient temperature, which generally affects the MEMS based devices. Further, the temperature variations during the day and also between day and night are always of concern for most of the in-situ soil moisture sensors, as the temperature variations may alter the sensor's response. For the in-situ measurements, the sensor's readings should be independent on the diurnal temperature variations to maintain the accuracy of the soil moisture measurements. Thus, the inventors have studied the effect of the temperature on the fabricated sensor 400 and analyzed the error introduced in the soil moisture measurements. To analyze the sensor response for different temperatures, the sensor has been placed in a chamber that has its temperature controlled and the temperature inside the chamber was varied from 15° C. to 60° C. and the humidity was maintained at 50% RH. FIG. 8 shows the response change (in terms of the relative capacitance) of the sensor 400 for these temperatures and the errors in the readings over this temperature range is smaller than 20%. Thus, in one embodiment, the processing unit 420 of the sensor 400 can be programmed to run a temperature correction software so that the reading of the sensor is adjusted based on the ambient temperature. If this approach is taken, a temperature sensor 450 may also be implemented in the sensor 400, as shown in FIG. 4. Thus, the processing unit 420 is configured to receive both moisture and temperature readings, and can adjust the moisture calculated value based on an initial calibration of the sensor 400, to take into account the temperature variation shown in FIG. 8.

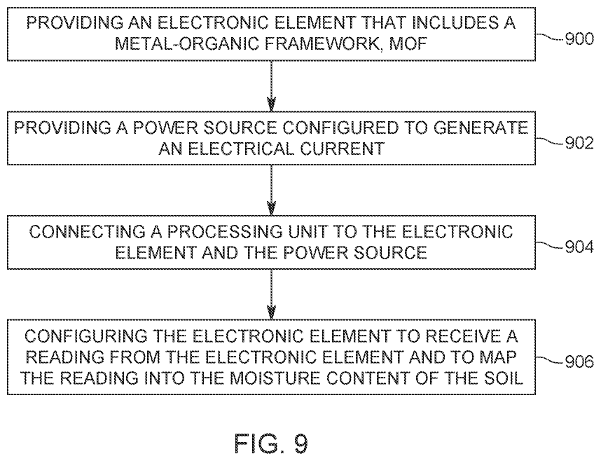

A method of making a moisture sensor for measuring a moisture content in soil is now discussed with regard to FIG. 9 and the method includes a step 900 of providing an electronic element that includes a metal-organic framework, MOF, a step 902 of providing a power source configured to generate an electrical current, a step 904 of connecting a processing unit to the electronic element and the power source, and a step 906 of configuring the electronic element to receive a reading from the electronic element and to map the reading into the moisture content of the soil.

In one application, the electronic element is a transistor and the MOF is located on a semiconductor layer, which extends over a source electrode and a drain electrode. In another application, the electronic element is a capacitor and the MOF is located over a source electrode and a drain electrode.

The disclosed embodiments provide a moisture sensor that uses a MOF for enhancing the sensitivity and selectivity to the moisture relative to other elements of the soil. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] S. J. Patil, A. Adhikari, M. S. Baghini, V. R. Rao, An ultra-sensitive piezo-resistive polymer nano-composite microcantilever platform for humidity and soil moisture detection, Sensors and Actuators B: Chemical, 203(2014) 165-73.
[2] T. Jackson, K. Mansfield, M. Saafi, T. Colman, P. Romine, Measuring soil temperature and moisture using wireless MEMS sensors, Measurement, 41(2008) 381-90.
[3] A. Quddious, S. Yang, M. M. Khan, F. A. Tahir, A. Shamim, K. N. Salama, K. N. and H. M. Cheema, Disposable, paper-based, inkjet-printed humidity and H2 S gas sensor for passive sensing applications. Sensors, 16(2016) 2073.
[4] M. A. Andres, M. T. Vijjapu, S. G. Surya, O. Shekhah, K. N. Salama, C. Serre, et al., Methanol and Humidity Capacitive Sensors Based on Thin Films of MOF Nanoparticles, ACS Applied Materials & Interfaces, (2020).

1. A moisture sensor configured to be deployed in soil for measuring a moisture content, the moisture sensor comprising:
a housing;
a transistor configured to interact with water from the soil;
a power source configured to generate an electrical current; and
a processing unit configured to receive a reading from the transistor, and to calculate the moisture content of the soil based on the reading,
wherein the transistor includes a metal-organic framework, MOF.

2. The moisture sensor of claim 1, wherein the MOF is located on a semiconductor layer, which extends over a source electrode and a drain electrode of the transistor.

3. The moisture sensor of claim 2, wherein the source and drain electrodes are interdigitated electrodes.

4. The moisture sensor of claim 1, wherein the MOF includes Zr, C, O, and H.

5. The moisture sensor of claim 1, wherein a chemical configuration of the MOF is Zr₆O₄OH₄(C₄H₂O)₆.

6. The moisture sensor of claim 2, wherein the MOF is directly located on the semiconductor layer.

7. The moisture sensor of claim 1, wherein the processing unit is configured to apply the electrical current to the transistor, and to calculate a change in a capacitance of the MOF.

8. The moisture sensor of claim 7, wherein the processing unit is further configured to calculate a relative change of the capacitance, and to associate the relative change of the capacitance with the moisture content of the soil.

9. A moisture sensor configured to be deployed in soil for measuring a moisture content, the moisture sensor comprising:
a housing;
a capacitor configured to interact with water from the soil;
a power source configured to generate an electrical current; and
a processing unit configured to receive a reading from the capacitor, and to calculate the moisture content of the soil based on the reading,
wherein the capacitor includes a metal-organic framework, MOF, as a dielectric material.

10. The moisture sensor of claim 9, wherein the MOF is located over a first and second electrodes.

11. The moisture sensor of claim 10, wherein the first and second electrodes are interdigitated electrodes distributed on a common substrate.

12. The moisture sensor of claim 10, wherein the MOF is in direct contact with the first and second electrodes.

13. The moisture sensor of claim 9, wherein the MOF includes Zr, C, O, and H.

14. The moisture sensor of claim 9, wherein a chemical configuration of the MOF is Zr₆O₄OH₄(C₄H₂O)₆.

15. The moisture sensor of claim 9, wherein the processing unit is configured to apply the electrical current to the capacitor, and to calculate a change in a capacitance of the MOF.

16. The moisture sensor of claim 15, wherein the processing unit is further configured to calculate a relative change of the capacitance, and to associate the relative change of the capacitance with the moisture content of the soil.

17. A method of making a moisture sensor for measuring a moisture content in soil, the method comprising:
providing an electronic element that includes a metal-organic framework, MOF;
providing a power source configured to generate an electrical current;
connecting a processing unit to the electronic element and the power source; and
configuring the electronic element to receive a reading from the electronic element and to calculate the moisture content of the soil based on the reading.

18. The method of claim 17, wherein the electronic element is a transistor and the MOF is located on a semiconductor layer, which extends over a source electrode and a drain electrode.

19. The method of claim 17, wherein the electronic element is a capacitor and the MOF is located over first and second electrodes.

20. The method of claim 17, wherein a chemical configuration of the MOF is Zr₆O₄OH₄(C₄H₂O)₆.
